(19) United States
(12) Patent Application Publication
(10) Pub. No.: US 2026/0334991 A1
(43) Pub. Date: Oct. 29, 2026

(54) STERNAL PLATE

(71) Applicant: Changzhou Waston Medical Appliance Co., Ltd, Changzhou (CN)

(72) Inventors: Renmin Hu, Changzhou (CN); Hong Liu, Changzhou (CN); Kejin Hang, Changzhou (CN)

(21) Appl. No.: 19/704,350

(22) Filed: Jun. 10, 2026

(30) Foreign Application Priority Data
Nov. 3, 2025 (CN) 202511593836.1

(51) Int. Cl. A61B 17/80

(52) U.S. Cl. CPC A61B 17/8076

(57) ABSTRACT

The present disclosure provides a sternal plate, which relates to the technical field of medical devices. The sternal plate includes a first connector and a second connector. The first connector includes an insertion plate with an insertion protrusion and first anti-slip teeth. The second connector has a connecting end including a first clamping portion, a second clamping portion, and a limiting groove matching the insertion plate. A clamping opening is formed between the clamping portions, with its side walls provided with second anti-slip teeth. The insertion plate cooperates with the limiting groove for guided insertion, and the insertion protrusion engages with the clamping opening to connect the two connectors. In emergencies requiring secondary sternotomy, the sternal plate can be cut open with common instruments, simplifying removal.

### CROSS-REFERENCE TO RELATED APPLICATIONS

The present application claims the benefit of Chinese Patent Application No. 202511593836.1 filed on Nov. 3, 2025, the contents of which are incorporated herein by reference in their entirety.

### TECHNICAL FIELD

The present disclosure relates to the field of medical device technology, and in particular to a sternal plate.

### BACKGROUND

Currently, in the medical field, sternal plates have largely replaced steel wires and have become the mainstream choice for sternal fixation. The basic structure of a sternal plate typically includes two connectors with clamping claw structures, and the clamping claw structures on these two connectors are arranged opposite to each other. During surgery, the sternum is approximated by these two clamping claw structures, and then fasteners are used to connect the two connectors, thereby achieving effective fixation of the sternum.

However, most of the existing sternal plates are made of titanium alloy. Due to the high strength characteristics of titanium alloy, special surgical tools and complex procedures are often required for removal. For patients who require a second sternotomy after an open-chest surgery or need rapid removal of the sternal plate, this material presents significant drawbacks. It increases surgical time and operational difficulty, bringing additional risks to the patients.

### SUMMARY

To address the issue of difficult removal of existing sternal plates during secondary sternotomy, the present disclosure provides a sternal plate. An insertion plate is adapted to cooperate with the limiting groove to achieve limiting and guiding during the insertion process, and the engagement between the insertion protrusion and the clamping opening is designed to connect the two connectors. In emergencies requiring secondary sternotomy, common instruments can be used directly for cutting the sternal plate, allowing for quick opening without specialized tools, thereby simplifying the surgical procedure.

The technical solution adopted by the present disclosure to solve the technical problem is as follows:

A sternal plate, including a first connector and a second connector;

the first connector includes an insertion plate; the insertion plate is provided with an insertion protrusion, and a plurality of first anti-slip teeth are arranged on both sides of the insertion protrusion along an insertion direction;

the second connector includes a connecting end;

the connecting end includes a first clamping portion, a second clamping portion, and a limiting groove adapted to the insertion plate, in which a clamping opening is formed between the first clamping portion and the second clamping portion, clamping opening side walls are arranged on opposite sides of the clamping opening, and each of the clamping opening side walls is provided with second anti-slip teeth; and

the insertion plate is adapted to cooperate with the limiting groove to achieve limiting and guiding during the insertion process; when the insertion protrusion enters the clamping opening, the first clamping portion and the second clamping portion elastically deform outward to allow the insertion protrusion to smoothly enter the clamping opening, and when the first anti-slip teeth and the second anti-slip teeth engage with each other, the elastic deformation is restored; connection between the first connector and the second connector is achieved by the engagement of the first anti-slip teeth with the second anti-slip teeth.

In some embodiments, the first anti-slip teeth are arranged in a vertical direction.

In some embodiments, the first anti-slip teeth are arranged at an angle of 20° to 60° relative to a vertical direction.

In some embodiments, the length of the clamping opening side walls in a horizontal direction is at least 10 mm.

In some embodiments, a tooth length of the first anti-slip teeth and the second anti-slip teeth is 1.5 mm to 2.5 mm.

In some embodiments, the connecting end further includes a reinforcement portion, and the reinforcement portion is arranged on the outer sides of the first clamping portion and the second clamping portion.

In some embodiments, a thickness of the reinforcement portion is 1.5 mm to 2 mm.

In some embodiments, the first anti-slip teeth and the second anti-slip teeth are sawtooth-shaped.

In some embodiments, the first anti-slip teeth and the second anti-slip teeth are strip-shaped tooth rows extending along the insertion direction, and a gap is provided between adjacent tooth rows, and the clamping opening side wall is provided with a notch adapted thereto.

In some embodiments, the sternal plate is further provided with a metal mark.

The present disclosure has the following beneficial effects:

The sternal plate provided by the present disclosure achieves a stable connection through the guiding cooperation between the limiting flange and the limiting groove, as well as the anti-slip engagement structure between the insertion protrusion and the clamping opening. It allows for flexible adjustment of the connection length according to the patient's sternal dimensions, significantly enhancing the applicability and versatility of the sternal plate. Secondly, the present disclosure mainly relies on the lateral deformation of the clamping portions on both sides to achieve fixation. By setting an angle between the clamping opening side walls and the vertical direction, the thickness of the plate body is reduced while ensuring its strength, effectively avoiding postoperative discomfort or foreign body sensation caused by an overly thick plate. In addition, the thinner plate reduces pressure on surrounding tissues, which is conducive to postoperative recovery and lowers the risk of complications. Furthermore, the sternal plate of the present disclosure is made of carbon fiber-reinforced modified PEEK material. In emergencies requiring secondary sternotomy, it can be directly cut using common instruments, enabling quick opening and removal without specialized tools. This simplifies the assembly and removal process of the sternal plate, greatly streamlining the surgical procedure.

### BRIEF DESCRIPTION OF DRAWINGS

The present disclosure will be further described below with reference to figures and embodiments.

FIG. 1 is a first assembly schematic diagram of a sternal plate of the present disclosure;

FIG. 2 is an exploded view of the sternal plate in FIG. 1;

FIG. 3 is a structural schematic diagram of the sternal plate in FIG. 1;

FIG. 4 is a second assembly schematic diagram of a sternal plate of the present disclosure;

FIG. 5 is a structural schematic diagram of the sternal plate in FIG. 4;

FIG. 6 is an enlarged schematic diagram of portion A in FIG. 4;

FIG. 7 is a third assembly schematic diagram of a sternal plate of the present disclosure;

FIG. 8 is a structural schematic diagram of the sternal plate in FIG. 7; and

FIG. 9 is an enlarged schematic diagram of portion B in FIG. 7.

In the figures:
1-first connector;
11-insertion plate;
111-insertion protrusion;
1111-first anti-slip teeth;
112-insertion head;
12-first clamping claw structure;
2-second connector;
21-connecting end;
211-limiting groove;
212-first clamping portion;
213-second clamping portion;
214-clamping opening;
215-clamping opening side wall;
2151-second anti-slip teeth;
2152-notch;
216-reinforcement portion;
22-second clamping claw structure;
3-metal mark.

### DETAILED DESCRIPTION OF THE EMBODIMENTS

The present disclosure will now be described in further detail. The embodiments described below are exemplary and are intended to explain the present disclosure, but should not be understood as limiting the present disclosure. Based on the embodiments of the present disclosure, all other embodiments obtained by ordinary technicians in the art without creative work belong to the scope of protection of the present disclosure.

In order to make the above objectives, features, and advantages of the present disclosure more obvious and understandable, specific embodiments of the present disclosure will be described in detail with reference to the drawings below.

It should be noted that, in the figures of the present disclosure, the arrow labeled "V" indicates the vertical direction, and the arrow labeled "H" indicates the horizontal direction.

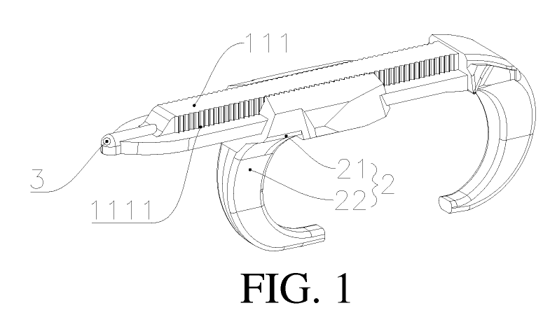

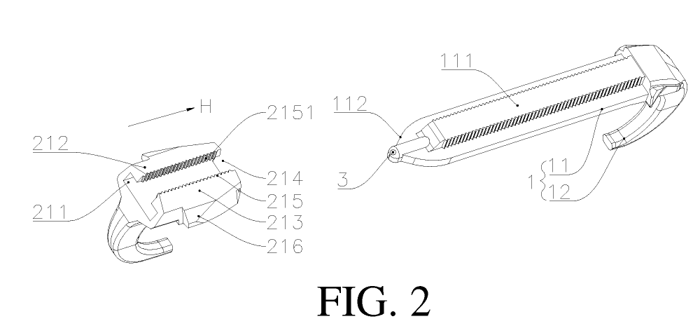

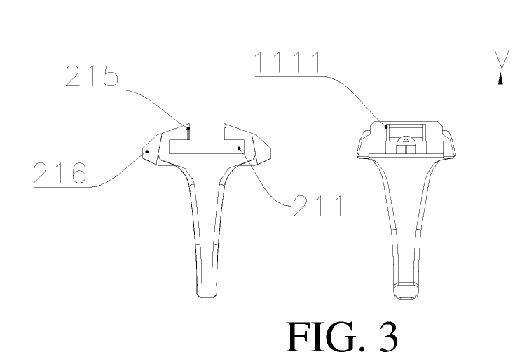

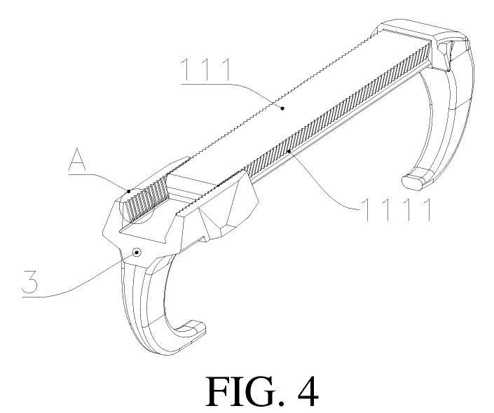

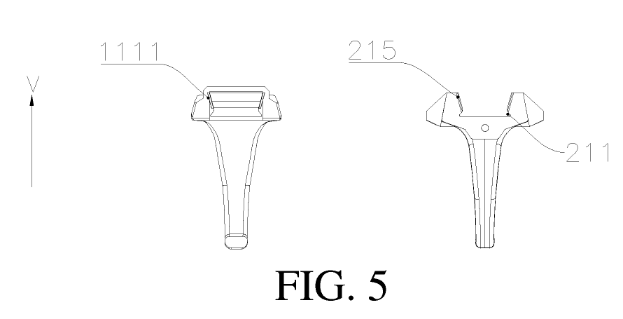

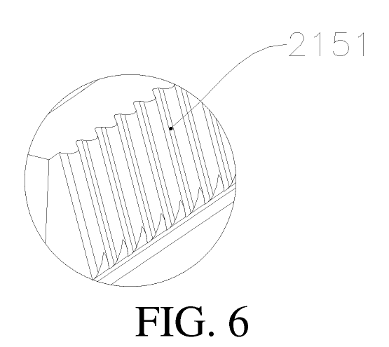

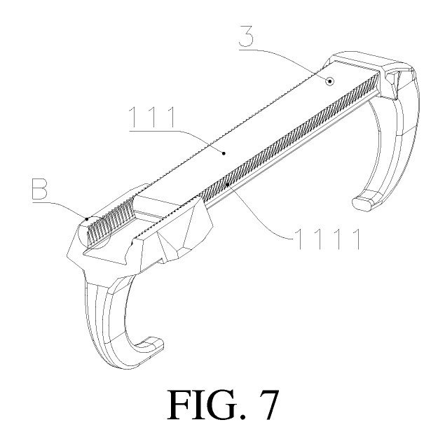

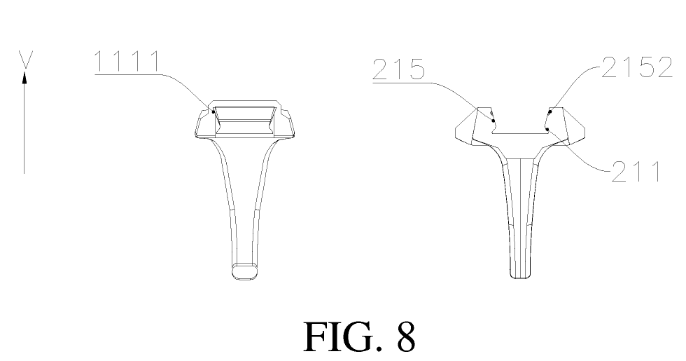

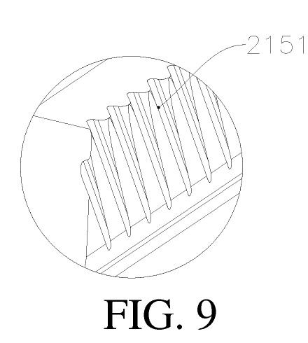

To solve the problem of difficult removal of sternal plates in the prior art, as shown in FIGS. 1–9, the present disclosure provides a sternal plate, including a first connector 1 and a second connector 2. The first connector 1 includes an insertion plate 11 and a first clamping claw structure 12. The insertion plate 11 is provided with an insertion protrusion 111, and a plurality of first anti-slip teeth 1111 are arranged on both sides of the insertion protrusion 111 along the insertion direction. The second connector 2 includes a connecting end 21 and a second clamping claw structure 22. The connecting end 21 includes a first clamping portion 212, a second clamping portion 213, and a limiting groove 211 adapted to the insertion plate 11. A clamping opening 214 is formed between the first clamping portion 212 and the second clamping portion 213. Clamping opening side walls 215 are provided on opposite sides of the clamping opening 214, and each of the clamping opening side walls 215 is provided with second anti-slip teeth 2151.

The insertion plate 11 is adapted to cooperate with the limiting groove 211 to achieve limiting and guiding during the insertion process; when the insertion protrusion 111 enters the clamping opening 214, the first clamping portion 212 and the second clamping portion 213 elastically deform outward to allow the insertion protrusion 111 to smoothly enter the clamping opening 214, and when the first anti-slip teeth 1111 and the second anti-slip teeth 2151 engage with each other, the elastic deformation is restored; connection between the first connector 1 and the second connector 2 is achieved by the engagement of the first anti-slip teeth 1111 with the second anti-slip teeth 2151.

To facilitate the approximation and fixation of the sternum, the present disclosure preferably configures the first clamping claw structure 12 and the second clamping claw structure 22 as hook-shaped structures suitable for approximating and fixing the sternum. After the first connector 1 is connected to the second connector 2, the first clamping claw structure 12 and the second clamping claw structure 22 are arranged opposite to each other, located on either side of the sternum respectively, forming a space therebetween suitable for accommodating the sternum, thereby achieving approximation and fixation of the sternum.

The sternal plate of the present disclosure is preferably made of carbon fiber-reinforced modified polyetheretherketone (PEEK). In emergencies requiring secondary sternotomy, common instruments can be used directly for cutting the sternal plate, enabling rapid opening and removal without specialized tools. This greatly simplifies the surgical procedure, shortens surgery time, and reduces surgery risks. By employing carbon fiber-reinforced modified PEEK material, the present disclosure avoids the issue of material adhesion to tissues, facilitating secondary surgery and reducing both surgery time and the risk of complications.

In addition, Chinese Patent Application Publication No. CN117281601A discloses a sternal plate with an insertion piece, whose structure relies on mutual engagement of upper and lower meshing structures, primarily achieving fixation through the longitudinal deformation of the clamping portions on both sides. To ensure sufficient gripping force, the clamping portions on both sides need to be made very thick. However, since the skin and muscle of the sternum are relatively thin, installing a thick sternal plate will require significant stretching of the muscles when suturing the skin and muscles. This not only increases the difficulty of the surgical procedure but can also cause the patient to experience strong foreign body sensation after surgery, and even cause complications.

To solve the above problems, the present disclosure preferably arranges the first anti-slip teeth in the vertical direction. Correspondingly, the clamping opening side walls 215 are also set in the vertical direction. This allows the fixation to rely primarily on the lateral deformation of the clamping portions on both sides. Consequently, there is no need to make the clamping portions very thick, while still ensuring the plate has sufficient gripping force and fixation strength.

To reduce the thickness of the clamping portions on both sides while ensuring the strength of the sternal plate, the present disclosure preferably sets the angle between the first anti-slip teeth 1111 and the vertical direction to 20°-60°. Correspondingly, the angle between the clamping opening side walls 215 and the vertical direction is also 20°-60°. Further, the present disclosure preferably sets the angle between the first anti-slip teeth 1111 and the vertical direction to 30°-45°. Within this angle range, a horizontal component force greater than the vertical component force can be achieved, thereby minimizing the thickness of the sternal plate while ensuring its strength.

To provide sufficient contact area and clamping force, the present disclosure preferably sets the length of the clamping opening side walls 215 in the horizontal direction to be at least 10 mm.

The present disclosure preferably sets the tooth length of the first anti-slip teeth 1111 and the second anti-slip teeth 2151 to 1.5 mm to 2.5 mm. This ensures the mechanical strength of the sternal plate while avoiding unreliable engagement caused by excessively thin teeth. It guarantees good stability and reliability of the sternal plate during use, while reducing the patient's foreign body sensation.

The sternal plate provided by the present disclosure achieves a stable connection through the guiding cooperation between the insertion plate 11 and the limiting groove 211, as well as the anti-slip engagement structure between the insertion protrusion 111 and the clamping opening 214. It allows for flexible adjustment of the connection length according to the patient's sternal dimensions, significantly enhancing the applicability and versatility of the sternal plate. Secondly, by setting an angle between the clamping opening side walls 215 and the vertical direction, the thickness of the plate body is reduced while ensuring its strength, effectively avoiding postoperative discomfort or foreign body sensation caused by an overly thick plate. In addition, the thinner plate reduces pressure on surrounding tissues, which is conducive to postoperative recovery and lowers the risk of complications. Furthermore, the sternal plate of the present disclosure is made of carbon fiber-reinforced modified PEEK material. In emergencies requiring secondary sternotomy, it can be directly cut using common instruments, enabling quick opening and removal without specialized tools. This simplifies the assembly and removal process of the sternal plate, greatly streamlining the surgical procedure.

To prevent fatigue damage in the outer regions of the first clamping portion 212 and the second clamping portion 213 caused by excessive stress, the present disclosure preferably includes a reinforcement portion 216 at the connecting end 21, and the reinforcement portion 216 is arranged on the outer side of the first clamping portion 212 and the second clamping portion 213 to enhance the structural strength and durability of the entire connecting end.

Furthermore, the present disclosure preferably sets the thickness of the reinforcement portion 216 to 1.5 mm to 2 mm, which can effectively resist fatigue damage caused by excessive stress while avoiding operational inconvenience that could arise from an overly thick reinforcement portion.

The present disclosure preferably configures the first anti-slip teeth 1111 and the second anti-slip teeth 2151 as sawtooth-shaped. This design can provide a stronger locking force and a more stable connection, thereby offering a better anti-slip effect during the insertion process, ensuring a more secure connection between the first connector 1 and the second connector 2, and reducing the possibility of loosening.

The present disclosure preferably arranges the first anti-slip teeth 1111 and the second anti-slip teeth 2151 as strip-shaped tooth rows extending along the insertion direction, with gaps provided between adjacent tooth rows. The clamping opening side wall is provided with a notch 2152 adapted thereto. This allows for adjustment of the insertion depth within a certain range to accommodate sternums of different thicknesses, improving the flexibility and adaptability of the insertion. In addition, the gaps help reduce friction during the insertion process, making assembly smoother.

To facilitate assembly, the present disclosure preferably provides an insertion head 112 at the front end of the insertion plate 11, i.e., the end of the insertion plate 11 away from the first clamping claw structure 12. This insertion head 112 can be triangular, arc-shaped, circular, rectangular, or have a similar structure.

It should be noted that after the first connector 1 is assembled with the first connector 2, the excess portion of the insertion plate 11 at the end where the insertion head 112 is located can be cut off. The specific cutting length is determined based on the dimensions of the sternum.

The sternal plate of the present disclosure is made of carbon fiber-reinforced modified PEEK material. Although this material has good biocompatibility and is radiolucent, it will be difficult to detect by imaging if the sternal plate accidentally falls off and migrates within the body. To address this issue, the present disclosure preferably further equips the sternal plate with a metal mark 3, facilitating the localization of the sternal plate within the body.

Taking the above exemplary embodiments of the present disclosure as guidance, a person skilled in the art can make various changes and modifications based on the above description, without departing from the scope and spirit of the present disclosure. The technical scope of the present disclosure is not limited to the content disclosed in the specification, but must be determined in accordance with the scope of the claims.

What is claimed is:

1. A sternal plate, comprising a first connector (1) and a second connector (2);
wherein the first connector (1) comprises an insertion plate (11); the insertion plate (11) is provided with an insertion protrusion (111), and a plurality of first anti-slip teeth (1111) are arranged on both sides of the insertion protrusion (111) along an insertion direction;
the second connector (2) comprises a connecting end (21);
the connecting end (21) comprises a first clamping portion (212), a second clamping portion (213), and a limiting groove (211) adapted to the insertion plate (11), wherein a clamping opening (214) is formed between the first clamping portion (212) and the second clamping portion (213), clamping opening side walls (215) are arranged on opposite sides of the clamping opening (214), and each of the clamping opening side walls (215) is provided with second anti-slip teeth (2151); and
the insertion plate (11) is adapted to cooperate with the limiting groove (211) to achieve limiting and guiding during the insertion process; when the insertion protrusion (111) enters the clamping opening (214), the first clamping portion (212) and the second clamping portion (213) elastically deform outward to allow the insertion protrusion (111) to smoothly enter the clamping opening (214), and when the first anti-slip teeth (1111) and the second anti-slip teeth (2151) engage with each other, the elastic deformation is restored; connection between the first connector (1) and the second connector (2) is achieved by the engagement of the first anti-slip teeth (1111) with the second anti-slip teeth (2151).

2. The sternal plate according to claim 1, wherein the first anti-slip teeth (1111) are arranged in a vertical direction.

3. The sternal plate according to claim 1, wherein the first anti-slip teeth (1111) are arranged at an angle of 20° to 60° relative to a vertical direction.

4. The sternal plate according to claim 1, wherein the length of the clamping opening side walls (215) in a horizontal direction is at least 10 mm.

5. The sternal plate according to claim 1, wherein a tooth length of the first anti-slip teeth (1111) and the second anti-slip teeth (2151) is 1.5 mm to 2.5 mm.

6. The sternal plate according to claim 1, wherein the connecting end (21) further comprises a reinforcement portion (216), and the reinforcement portion (216) is arranged on the outer sides of the first clamping portion (212) and the second clamping portion (213).

7. The sternal plate according to claim 6, wherein a thickness of the reinforcement portion (216) is 1.5 mm to 2 mm.

8. The sternal plate according to claim 4, wherein the first anti-slip teeth (1111) and the second anti-slip teeth (2151) are sawtooth-shaped.

9. The sternal plate according to claim 4, wherein the first anti-slip teeth (1111) and the second anti-slip teeth (2151) are strip-shaped tooth rows extending along the insertion direction, and a gap is provided between adjacent tooth rows, and the clamping opening side wall (215) is provided with a notch (2152) adapted thereto.

10. The sternal plate according to claim 1, wherein the sternal plate is further provided with a metal mark (3).
